(19) United States
(12) Patent Application Publication
(10) Pub. No.: US 2016/0331408 A1
(43) Pub. Date: Nov. 17, 2016

(54) Atraumatic Cervical Tenaculum

(71) Applicant: Benson Medical LLC, Deerfield, IL (US)

(72) Inventors: Michael Benson, Deerfield, IL (US); Julia Benson, Deerfield, IL (US); Bonnie Benson, Deerfield, IL (US)

(21) Appl. No.: 15/153,344

(22) Filed: May 12, 2016

Related U.S. Application Data
(63) Continuation-in-part of application No. 29/549,722, filed on Dec. 28, 2015.
Application No. 29/549,722 is a continuation-in-part of application No. 29/549,713, filed on Dec. 28, 2015.
Application No. 29/549,713 is a continuation-in-part of application No. 29/554,174, filed on Feb. 9, 2016.
(60) Provisional application No. 62/161,343, filed on May 14, 2015.
(60) Provisional application No. 62/290,197, filed on Feb. 2, 2016.

(51) Int. Cl. A61B 17/42; A61B 17/28

(52) U.S. Cl. CPC A61B 17/42; A61B 17/2812; A61B 17/282; A61B 2017/4225; A61B 2017/2837; A61B 2017/00438

(57) ABSTRACT

A cervical tenaculum has first and second arms with respective proximal and a distal ends. The first arm and second arms are rotatably connected between the respective proximal and distal ends so that the distal ends form an arm plane. The cervical tenaculum forms a user portion at the proximal ends of the first and second arms, and has first and second projections respectively extending from substantially the distal end of the first and second arms. These projections each have respective substantially linear portions that diverge from the arm plane. To grasp cervical tissue, the cervical tenaculum has first and second footpads respectively coupled with the first and second projections. The first and second footpads define a grasping region for grasping cervical tissue.

### PRIORITY

This patent application claims priority from the following patent applications:
Provisional U.S. Patent Application No. 62/161,343, filed May 14, 2015, entitled “ATRAUMATIC TENACULUM,” and naming Michael Benson and Julia Benson as inventors (attorney docket no. 4166/1001),
Provisional U.S. Patent Application No. 62/290,197, filed Feb. 2, 2016, entitled “ATRAUMATIC TENACULUM,” and naming Michael Benson, Julia Benson and Bonnie Benson as inventors (attorney docket no. 4166/1005),
   and is a continuation-in-part application of the following design patent applications:
U.S. Design patent application Ser. No. 29/549,722, filed Dec. 28, 2015, entitled “TENACULUM,” and naming Michael Benson and Julia Benson as inventors (attorney docket no. 4166/1002),
U.S. Design patent application Ser. No. 29/549,713, filed Dec. 28, 2015, entitled “TENACULUM,” and naming Michael Benson and Julia Benson as inventors (attorney docket no. 4166/1003), and
U.S. Design patent application Ser. No. 29/554,174, filed Feb. 9, 2016, entitled “TENACULUM,” and naming Michael Benson and Julia Benson as inventors (attorney docket no. 4166/1006). The disclosures of all of the above patent applications are incorporated herein, in their entireties, by reference.

### FIELD OF THE INVENTION

The invention generally relates to gynecological instruments and, more particularly, the invention relates to a cervical tenaculum for grasping cervical tissue.

### BACKGROUND OF THE INVENTION

A single tooth tenaculum is a surgical instrument that allows a clinician to grasp the uterine cervix during gynecological procedures. The cervix contains a small canal directly into the uterine cavity that is accessible during pelvic examinations of women. There are a number of gynecologic procedures that require access to the uterine cavity, including:
Placement of intrauterine device (for contraception),
Dilatation and evacuation (procedure for emptying the uterus both during an abortion and for a miscarriage),
Biopsy of the uterine lining (for fertility and testing for cancer),
Dilatation and curettage (diagnostic procedure for both fertility and cancer testing), and
Hysteroscopy (operative and diagnostic).

These procedures may be done individually, in combination, or with other procedures/surgeries involving the female anatomy.

In aggregate, these procedures are performed several million times per year in the United States. As they typically involve pushing something through the cervix into the uterus, these procedures require a method for counter-traction on the cervix. That is, the cervix needs to be grasped, held in place with an instrument, and pulled toward the clinician as other instrument(s) is/are pushed through the cervix.

### SUMMARY OF VARIOUS EMBODIMENTS

In accordance with one embodiment of the invention, a cervical tenaculum has first and second arms with respective proximal and distal ends. The first arm and second arms are rotatably connected between the respective proximal and distal ends so that the distal ends form an arm plane. The tenaculum forms a user portion at the proximal ends of the first and second arms, and has first and second projections respectively extending from substantially the distal end of the first and second arms. These projections each have respective substantially linear portions that diverge from the arm plane. To grasp cervical tissue, the tenaculum has first and second footpads respectively coupled with the first and second projections. The first and second footpads define a grasping region for grasping cervical tissue.

The first footpad and the second footpad may be arcuate, among other shapes. Similar to an arc, the footpad may have a first end and a second end. The footpads may be coupled to the projections between the first end and the second end of the footpads. The footpads may have tissue contact surfaces, and the tissue contact surfaces may include at least one grasping feature, such as ridges. Additionally, the tenaculum may have a safety stop, so that a gap exists between the footpads even in a fully closed mode.

In some embodiments, the positions of the first footpad on the first projection and the second footpad on the second projection are fixed. Alternatively, the first footpad may be rotatably coupled with the first arm, and the second footpad may be rotatably coupled with the second arm. Furthermore, the first projection may be rotatably coupled with the first arm, and the second projection may be rotatably coupled with the second arm. Specifically, the angles formed between the arms and the substantially linear portions of the projections may be adjustable. Additionally, the lengths of the projections and/or arms may be adjustable.

Some embodiments may include a proximal plane formed by the proximal ends of the arms. The proximal plane may be formed at least in part by the user portion. The proximal plane may also diverge from the above mentioned arm plane. The footpads may be offset from the arm plane. Specifically, the footpads may not intersect the arm plane. In some embodiments, the footpads may be offset by between about 0.5 centimeters and about 2 centimeters.

The tenaculum may have a locking mechanism configured to prevent widening or narrowing of the grasping region when engaged. The locking mechanism may be disengaged to permit widening or narrowing of the grasping region. In some embodiments, the arm of the tenaculum may form an angle of between about 10 and 20 degrees.

In accordance with another embodiment, a cervical tenaculum has a first arm with a proximal end and a distal end. The tenaculum also has a second arm with a proximal end and a distal end. The first arm and the second arm are rotatably connected between the respective proximal and distal ends of the first and second arms. A user portion is at the proximal ends of the first and second arms. A first projection extends from substantially the distal end of the first arm, and a second projection extends from substantially the distal end of the second arm. A first footpad is coupled with the first projection, and a second footpad coupled with the second projection. The first and second footpads may have opposing surfaces with grasping features for grasping cervical tissue.

The tenaculum may include, among other things, a locking mechanism. The locking mechanism is configured to prevent rotation of the first arm relative to the second arm when engaged. Formed at the distal ends of the arms is an arm plane. The first projection may have a first substantially linear portion that diverges from the arm plane, and the second projection may have a second substantially linear portion that diverges from the arm plane. The arms of the tenaculum may form an angle of between about 10 and 20 degrees. A second plane may be formed by the arms.

In accordance with another embodiment of the invention, a method of examining a cervix provides a cervical tenaculum. The cervical tenaculum includes a first arm having a proximal end and a distal end and a second arm having a proximal end and a distal end. The first arm and the second arm are rotatably connected between the proximal and distal ends of the first and second arms to form an arm plane extending from the distal end. The tenaculum also includes a user portion at the proximal ends of the first and second arms. A first projection extends from substantially the distal end of the first arm, and a second projection extends from substantially the distal end of the second arm. A first footpad is coupled with the first projection, and a second footpad coupled with the second projection. The method grasps cervical tissue by rotating the first arm relative to the second arm in the arm plane to move the first footpad closer to the second footpad to grasp the cervical tissue.

The method may further engage a locking mechanism to prevent changing the distance between the arcuate footpads by preventing rotation of the first arm relative to the second arm. The tenaculum may be used to apply a force to the cervical tissue while positioning a second tool into the cervix. The first and second arms may form an arm plane extending from the distal ends of the first and second arms, and the first projection may have a first substantially linear portion that diverges from the arm plane.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows a perspective view of a cervical tenaculum configured in accordance with illustrative embodiments of the invention.

FIG. 2 schematically shows the cervical tenaculum of FIG. 1 grasping a patient's cervix.

FIG. 3 schematically shows a side view of the cervical tenaculum of FIG. 1.

FIG. 4 schematically shows a top view of the cervical tenaculum of FIG. 1.

FIG. 5 schematically shows a front view of the cervical tenaculum of FIG. 1.

FIG. 6 schematically shows a detail of a portion of the cervical tenaculum in FIG. 4.

FIG. 7 schematically shows a perspective view of an alternative embodiment of the cervical tenaculum in accordance with illustrative embodiments of the invention.

FIG. 8 shows a process of using the cervical tenaculum in accordance with illustrative embodiments of the invention.

### DESCRIPTION OF ILLUSTRATIVE EMBODIMENTS

In illustrative embodiments, a cervical tenaculum has increased contact surface area for less traumatically gripping cervical tissue. This increased surface area better distributes, and thus reduces, pressure experienced by cervical tissue. To that end, illustrative embodiments have high surface area arcuate footpads mounted on projections. The arcuate shape of the footpads approximates the shape of the cervical tissue, further enhancing pressure distribution, patient comfort, and grip strength. By mounting the footpads on projections, medical practitioners (e.g., gynecologists) have better visibility of, and access to, the cervix during medical procedures. Details of illustrative embodiments are discussed below.

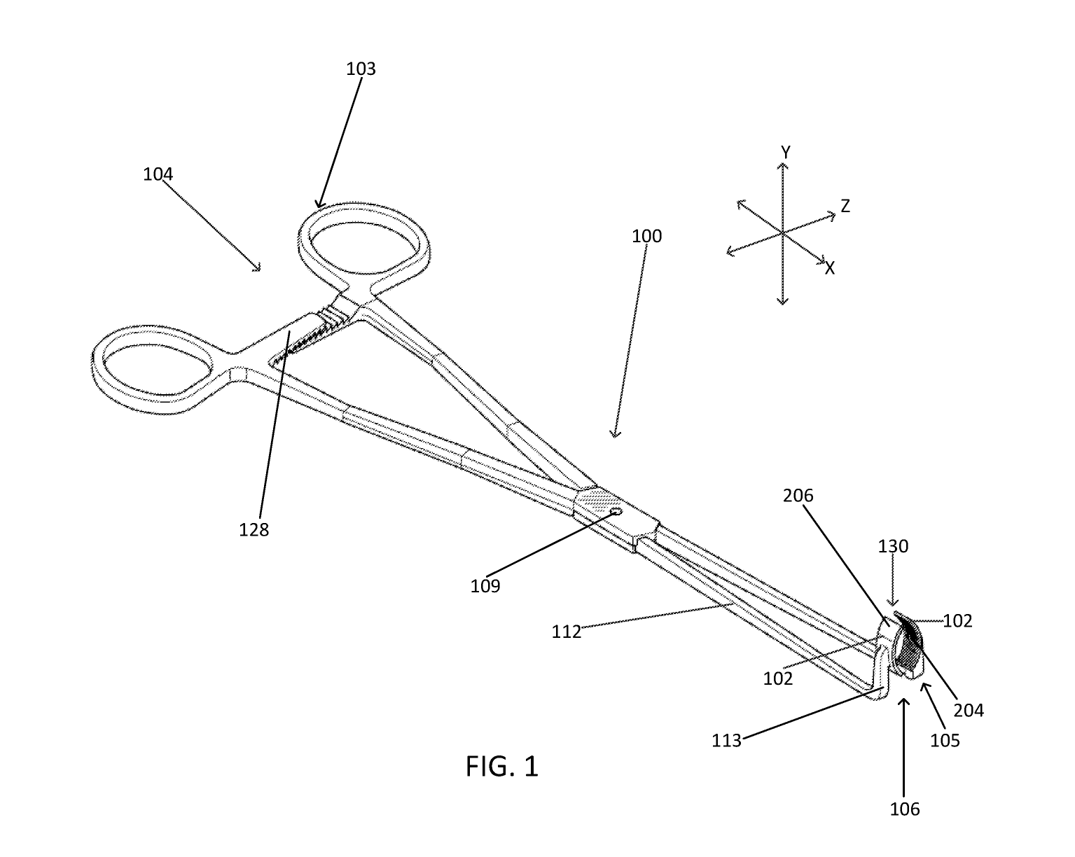

FIG. 1 schematically shows a perspective view of a cervical tenaculum (hereinafter “tenaculum 100”) configured in accordance with illustrative embodiments of the invention. At a high level, during use, a medical practitioner handles a proximal end 104 that defines a user portion 103. In a similar manner to conventional scissors, the medical practitioner manipulates the user portion 103 to relay corresponding movement to a distal end 106 via rotatably coupled arms 112. In illustrative embodiments, movement from the user portion 103 is relayed to the distal end 106 of the arms 112 about a pivot point 109. A corresponding grasping region 105 configured to grasp cervical tissue during use thus is positioned at the distal end 106, or substantially near the distal end 106. Thus, when the practitioner manipulates the user portion 103, corresponding movement is also produced at the grasping region 105.

More specifically, the practitioner manipulates the user portion 103 to enlarge or reduce the size of the grasping region 105 (discussed below). A person of skill in the art should understand that the user portion 103 does not necessarily have to be operated by a human practitioner; it may also be operated by a machine (e.g., robot). In illustrative embodiments, the grasping region 105 is formed by two similarly configured grasping members 102. As discussed below, the two grasping members 102 have inner surfaces 204 that together form a volume defining the grasping region 105. The distance between these two facing surfaces (referred to as a “gap 130”) thus changes as the practitioner manipulates the user portion 103. It is this gap 130 that can be enlarged/widened or reduced/narrowed. Accordingly, by manipulating the user portion 103, the practitioner may widen and/or narrow the gap 130 between the two grasping members 102.

The grasping members 102 are configured to efficiently and less traumatically grasp cervical tissue. In illustrative embodiments, as shown in FIG. 1, the grasping members 102 (also referred to as “pads 102” or “footpad(s) 102”) may take on any of a variety of shapes, such as an arcuate shape. The arcuate footpads 102 may have inner surfaces 204 and outer surfaces 206. Compared to prior art cervical tenaculums known to the inventors, the footpads 102 allow the tenaculum 100 to grasp increased cervical tissue surface area. In preferred embodiments, the footpads 102 grasp tissue by compressing it, not piercing it. To that end, the footpads 102 have real, appreciable contact surface area, and are not finely manufactured or blunt points, which often cause patient bleeding and other trauma. In some embodiments, the footpads 102 may be configured to contact when fully closed. However, the completely closed mode (i.e., no minimum gap) does not require that the footpads 102 make contact because when grasping tissue, tissue is between the footpads 102. Completely closing the footpads 102 therefore could, in some circumstances, undesirably damage certain tissues (e.g., crush or pierce the cervix).

The tenaculum 100 may formed from any suitable material conventionally used for such purposes. For example, among other things, the tenaculum 100 may be formed from metal, steel (e.g., stainless steel), plastic, or a combination thereof. Some embodiments also may have a polymer coating (e.g., a two-shot process).

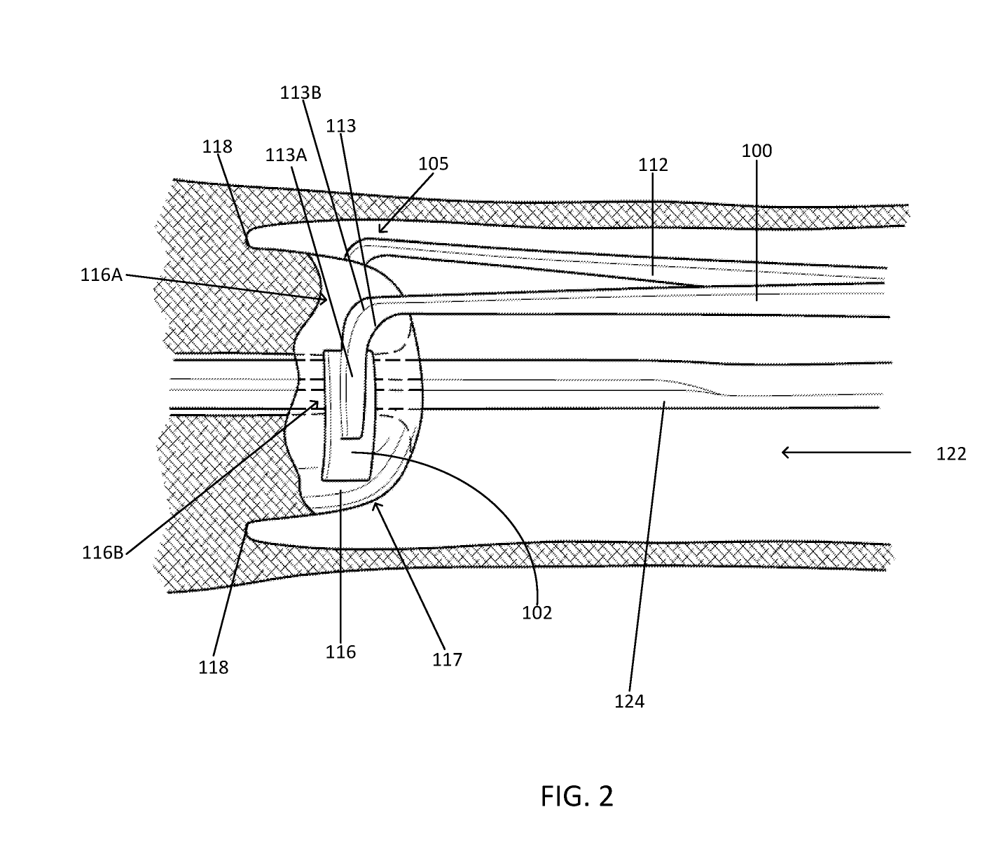

FIG. 2 schematically shows the tenaculum 100 of FIG. 1 grasping a patient's cervix 116. Frequently, the grasped cervical tissue 116 is the vaginal portion of the cervix 116, also known as the “ectocervix” and identified by reference number 117. The ectocervix 117 bulges through the wall 118 of the vagina, and therefore, is the portion of the cervix 116 most accessible to the practitioner.

Typically, the tenaculum 100 is moved distally into the vaginal canal 122 until the grasping region 105 contacts the cervix 116. For example, the practitioner may manipulate the user portion 103 to widen the grasping region 105, and place at least a portion of the cervix 116 within the gap 130 formed by the grasping region 105. The user portion 103 then may be manipulated to narrow the gap 130, grasping the cervix 116. Specifically, arcuate footpads 102 grasp the cervix 116 by contacting and compressing the cervical tissue 116. After grasping the cervical tissue 116 (e.g., the ectocervix 117), the practitioner may hold the cervix 116 steady, and instruments (e.g., a dilator) can be pushed distally—i.e., through the cervix 116 and distally of the tenaculum 100. In some embodiments, the practitioner can apply a force on the cervix 116 directed substantially towards the opening of the vagina.

Because of limited space and visibility, positioning the tenaculum 100 and a second medical instrument (e.g., dilator 124) within the vaginal canal 122 and/or cervix 116 can be challenging for a medical practitioner. Indeed, practitioners frequently insert a speculum into the vagina to separate the vaginal walls so that the cervix can be examined. Because of the limited space, the dilator 124 and the tenaculum 100 often are positioned into the vagina in generally parallel orientations, as shown. During such procedures, the inventors discovered that the visibility of the cervix 116, from the perspective of the practitioner, could be enhanced by physically offsetting the footpads 102 of the tenaculum 100 from the arms 112. This offset allows the footpads 102 to contact the cervix 116 without the arms 112 obstructing the central axis of the cervix 116. Accordingly, illustrative embodiments mount the footpads 102 on projections 113 extending from the arms 112. To that end, in illustrative embodiments, the projections 113 may have a substantially linear portion 113A, in addition to a curved attachment portion 113B that transitions into the substantially linear portion 113A. Other embodiments may have projections 113 oriented in other ways. Offsetting the footpads 102 on the projections 113 favorably leaves the central axis of the cervix 116 substantially unobstructed, enabling practitioners to more easily grip the cervical tissue 116. Furthermore, offsetting the footpads 102 provides the practitioner with improved access to the cervical canal (e.g., easier to insert uterine dilator 124 into the cervix 116).

In embodiments having footpads 102 mounted directly on the arms 112 (i.e., rather than on projections 113), it may be difficult for the tenaculum 100 to grasp the central portion of the cervix 116A—it typically grasps the top portion of the cervix 116A. In contrast, illustrative embodiments having the projections 113 may more easily enable the practitioner to grasp a central portion of the cervix 116B. In fact, without the projections 113, grasping the central portion of the cervix 116B may be impractical for some medical procedures. This is because, during these procedures, tools (e.g., dilator 124) are frequently positioned into the opening of the cervix 116, which is located at the central portion of the cervix 116B. Accordingly, grasping the central portion of the cervix 116B without mounting the footpads 102 on projections 113 may obstruct (e.g., arms 112) the passage of other tools through the cervical canal, which is also located as the central portion of the cervix 116B. Additionally, because of the limited space within the vaginal canal 122 and the vaginal opening, it is often impractical to insert the tenaculum 100 without projections 113 at an angle to grasp the central portion of the cervix 116B without obstructing access to the cervical canal.

It should be understood that the tenaculum 100 is shown in one of many possible orientations relative to the cervix 116. Illustrative embodiments of the tenaculum 100 could be inserted and/or rotated differently relative to the configuration shown in FIG. 2. Additionally, a second tenaculum 100 could be inserted and/or used to grasp cervical tissue 116. The second tenaculum 100 could be at a 90 degree offset from the shown tenaculum 100.

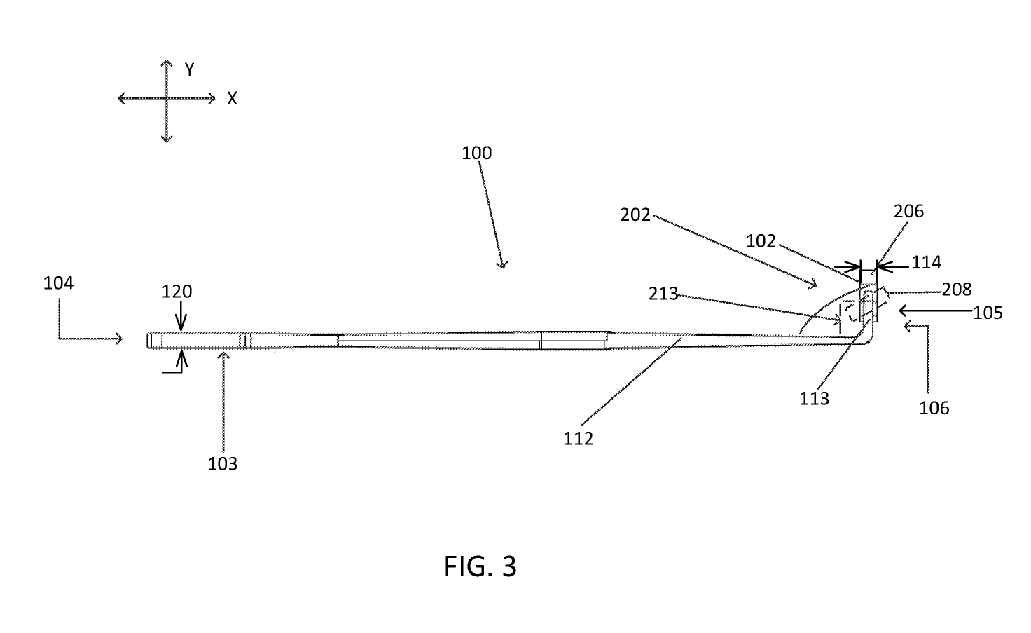

FIG. 3 schematically shows a side view of the tenaculum 100 of FIG. 1. In illustrative embodiments, the tenaculum 100 has a generally uniform thickness 120 at and near the proximal end 104 (e.g., about 4 millimeters). This thickness 120 may taper, however, toward the distal end 106. In some embodiments, the projections 113 are positioned substantially at the distal end 106. For example, the projections 113 may be at the actual end of the arms 112, as illustrated. However, in some other embodiments, the projections 113 may be near, or substantially at, the distal end 106 (e.g., slightly proximally positioned from the distal end).

As noted above, the projections 113 provide a physical offset for the footpads 102 so that the tenaculum 100 does not obstruct the central axis of the cervix 116. Particularly, the projections 113 may prevent the distal end 106 of the arms 112 from obscuring a practitioner's view of the grasping region 105. The projections 113 nevertheless do not necessarily have to be at the end of the arms 112. Instead, the projections 113 may be mounted to the arms 112 at a position proximal to the distal end 106. Accordingly, because of their connection to the projections 113, the footpads 102 may be spaced from the arms 112 in the X, Y, or Z-dimension, and/or in any combination thereof. This spacing may provide better access to tissue that is otherwise not easily accessible.

In illustrative embodiments, the projections 113 are fixedly mounted to the arms 112 at about a 90 degree projection angle 213. Other embodiments may fixedly mount the projections 113 at another angle, such as 85 degrees or less. Some embodiments, however, may movably/rotatably mount the projections 113 to the arms 112 about a projection pivot point. To that end, in some embodiments, at least a portion of the projections 113 may be rotatable to adjust the angle 213. Accordingly, prior to use, practitioners can adjust the angle 213 to best accommodate their needs. Indeed, in some embodiments, the projections 113 may be rotated to extend substantially below the tenaculum 100 (from the perspective of FIG. 3), rather than above the tenaculum 100. The angle 213 of the rotatably coupled projections 113 can be locked and unlocked, allowing the practitioner to adjust and secure the orientation of the projections 113.

In a similar manner, the orientation of the footpads 102 may be adjustable. For example, footpad angle 202 of FIG. 3 is approximately 90 degrees. However, the footpads 102 may be rotatably coupled to projections 113 to rotate about an axis, such as the Z-axis. For example, the footpads 102 may be rotated to an adjusted position 208 shown in broken lines of FIG. 3. Similar to the projections 113, the orientation of the footpads 102 may be locked and unlocked, allowing the practitioner to adjust and secure the orientation of the footpads 102. In other embodiments, the footpads 102 may be fixed (i.e., not rotatable) at a different angle 202 (e.g., about 45 degrees as shown in adjusted position 208).

As a physical object, the footpads 102 have a width 114 (e.g., about 6 millimeters). Wider footpads 102, which may have more cervical contact area than narrower footpads 102, also should provide a larger inner surface 204 to contact the cervical tissue. Some embodiments, however, may have a smaller inner surface area 204.

The footpads 102 may be coupled to the projections 113 in a number of ways. For example, in the embodiment of FIG. 1, the footpads 102 are mounted to their respective projections 113 on at least a portion of their outer surfaces 206. It therefore is not necessary for the footpads 102 to be mounted on the ends of the projections 113 (i.e., their ends are the maximum and minimum Y-axis points from the perspective of FIG. 3). Other embodiments, however, may mount the footpads 102 on the projections 113 at or very near the respective ends of the footpads 102, or even the respective sides of the footpads. The footpads 102 thus may be mounted to the projections 113 along any part of the outer surface 206 (e.g., between the ends of the footpad 102). In some other embodiments, footpads 102 may be mounted at their inner surface 204, although such an arrangement may undesirably obstruct grasping of the cervix 116. Additionally, or alternatively, the footpads 102 may be mounted to projections 113 at their thickness, i.e., between the inner surface 204 and outer surface 206.

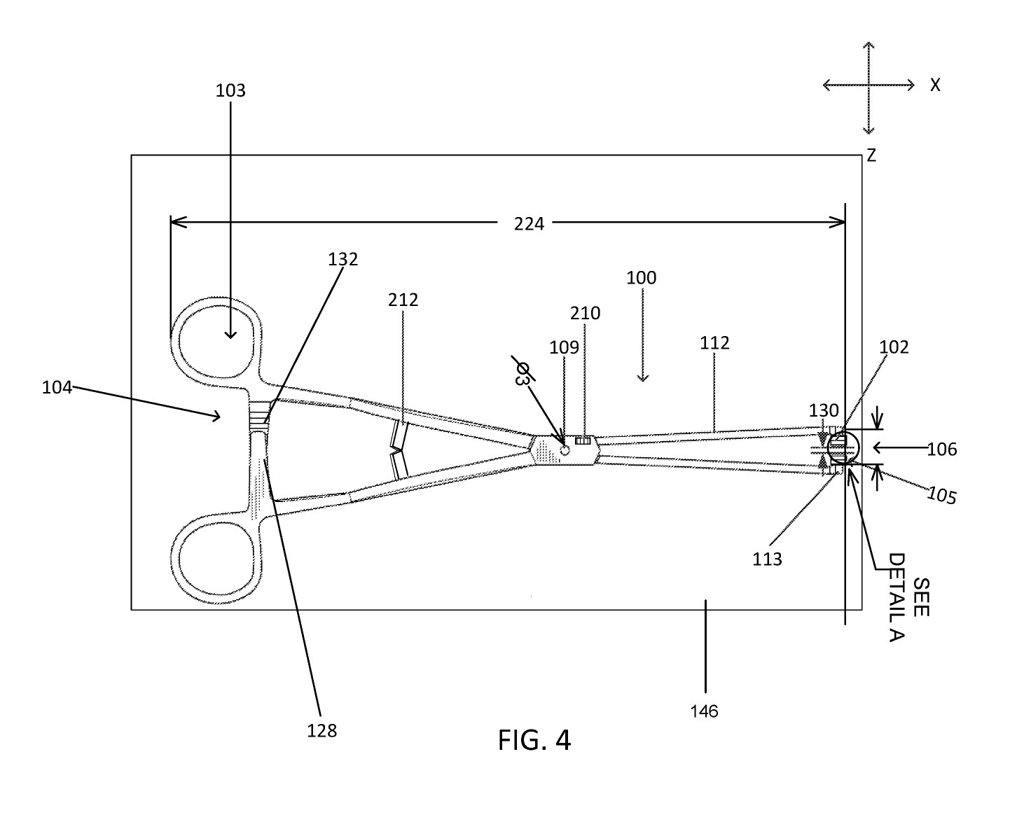

FIG. 4 schematically shows a top view of the tenaculum 100 of FIG. 1. As described above, the grasping region 105 may be widened and/or narrowed by user manipulations at the user portion 103. As noted above, widening and/or narrowing the grasping region 105 makes the gap 130 larger and/or smaller, respectively. A person of skill in the art will understand that certain dimensions of the tenaculum 100 may vary as the tenaculum 100 opens or closes. For example, as the tenaculum 100 opens, it will become shorter, but wider. For example, when the gap 130 is about 2.3 millimeters, the body of the tenaculum 100 may have a length 224 of about 25 centimeters, and a length from the distal end 106 to the pivot point 109 of about 105 millimeters. Additionally, the width of the grasping region 105 may be about 16 millimeters when the gap 130 is approximately 2.3 millimeters. Some embodiments have an adjustable length 224. To that end, the arms 112, for example, might be retractable or extendable.

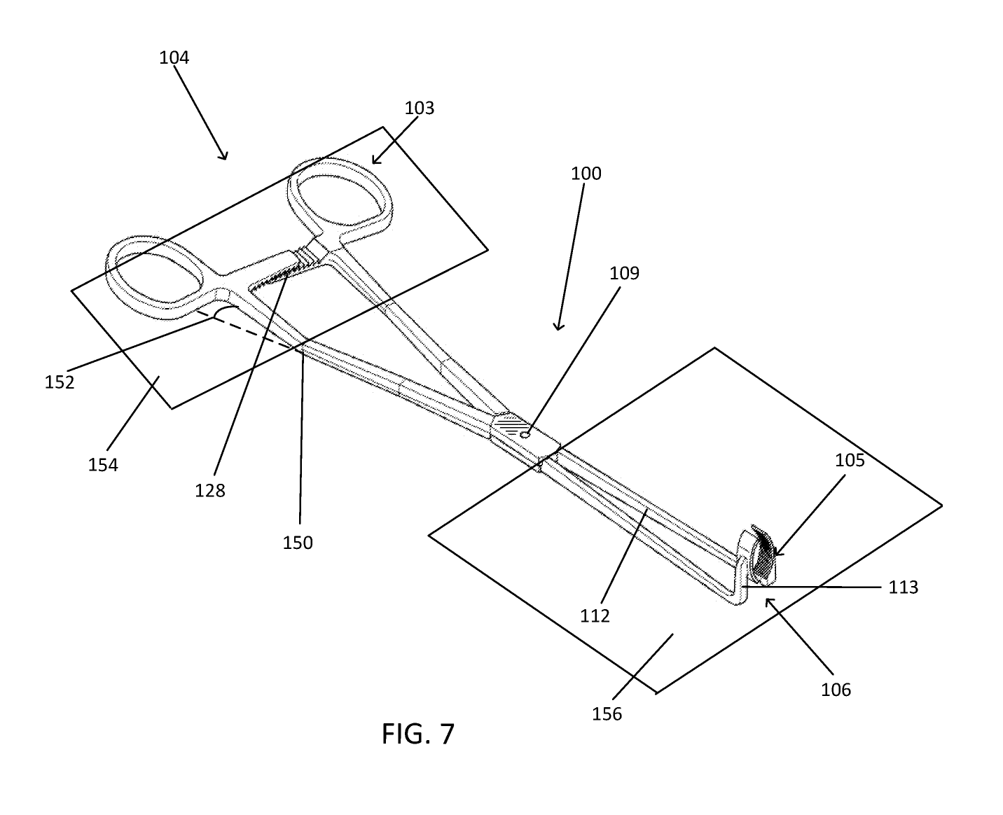

Additionally, the dimensions provided are merely exemplary and not intended to limit various embodiments of the invention. It should be understood that illustrative embodiments of the tenaculum 100 may come in various sizes (e.g., small adult, large adult, child, etc.). For example, some embodiments may involve a family of tenaculums 100. Each family may have a different size based on the radius or diameter of the footpads 102 (e.g., 1 cm, 1.5 cm, and 2 cm). Families may be categorized based on other criteria, however, such as the planarity of the arms 112 (see below discussion with regard to FIG. 7).

The tenaculum 100 may be considered to have a closed mode and an open mode. In illustrative embodiments, the tenaculum 100 is in the closed mode when the gap 130 between the footpads 102 is as small as allowed by the tenaculum 100. In some embodiments, the closed mode may have a gap 130 of zero (e.g., the footpads 102 touching). The tenaculum 100 thus is considered to be in the open mode when not in its closed mode.

The tenaculum 100 is expected to be used in a variety of gynecological procedures, and patients are not always anesthetized. To enhance patient comfort, some embodiments may have a safety stop 212—i.e., a closed mode with a minimum gap 130 (i.e., the closest distance between the footpads 102). In other words, the footpads 102 may not contact one another in the closed mode in the absence of grasped tissue. For example, in illustrative embodiments having arced footpads 102, the closest distance between the footpads 102 may be the distance between the ends of the arcs. Allowing the footpads 102 to completely close may, in some instances, cause pain to the patient and bleeding of the grasped tissue. This may be deterred by providing a minimum gap 130 between the footpads 102. In some embodiments, the minimum gap 130 may be about 2.3 centimeters (i.e., the footpads 102 can only come as close as 2.3 centimeters before the safety stops 212 prevents further closure of the footpads 102). However, the minimum gap 130 in various embodiments may vary. For example, the minimum gap 130 may be between about 0 millimeters and about 50 millimeters. Preferably, the minimum gap 130 is between about 5 millimeters and about 30 millimeters. In some embodiments, a spring formed from spring steel is used to maintain the gap 130 in the closed mode. Other embodiments may use a movable stiff component to inhibit rotation of the arms 112 relative to one another.

The average width of an adult ectocervix 117 is approximately 2.5 centimeters, although this can vary, particularly with childbirth experience. Furthermore, childrens' cervices might be smaller. However, the width of the ectocervix 117 may be up to 4 centimeters in some cases. Because every patient is different and practitioners may require different amounts of traction on the cervix 116 depending on the demands of the medical procedure, the practitioner may wish to adjust the minimum gap 130. To that end, some embodiments may have an adjustable minimum gap 130. For example, the tenaculum 100 may have an externally accessible gear 210, or wheel 210, configured to be adjusted by a user's thumb. Rotating the gear 210 adjusts the size of the minimum gap 130. For example, rotating the gear 210 may adjust the movable stiff component to allow further rotation of the arms 112 relative to one another. The minimum gap 130 can be adjusted from fully closed (e.g., 0 centimeters gap 130) up to a 5 centimeters gap 130 in increments of, for example, 1 millimeter. The practitioner can adjust the gap 130 to take into account tissue type and dimensions, as well as user comfort. In some embodiments, the thumb gear 210 may be located near the pivot point 109. Additionally, or alternatively, the tenaculum 100 may come with a preset minimum gap 130.

While the gap 130 is intended to minimize or prevent excessive force on the tissue, the grasping region 105 still applies force to the cervical tissue 116. To better control this force, some embodiments of the tenaculum 100 also have a locking mechanism 128 to retain a constant gap 130 (e.g., FIGS. 1 and 4). Accordingly, when the tenaculum 100 grasps the ectocervix 117, the practitioner may engage the locking mechanism 128, preventing the gap 130 from widening (or getting smaller) to ensure a steady force. Accordingly, the practitioner no longer has to constantly apply force to keep the gap 130 steady and retain the cervical tissue 116. Among other things, the locking mechanism 128 may comprise a series of interlocking teeth 132 located near the user portion 103. The practitioner may disengage the locking mechanism 128 by causing the interlocking teeth 132 to move apart. Other embodiments may use other types of locking mechanisms, such as fasteners or buttons/holes. Accordingly, discussion of the interlocking teeth 132 is but one embodiment of the locking mechanism 128.

The tenaculum 100 may be considered to form or lie along one or more planes 146. The side view of FIG. 3, for example, shows a significant majority of the tenaculum 100 as lying along a single plane 146, and its projections 113 extending along another plane that is generally normal to the noted single plane 146. Accordingly, in the example of FIG. 3, the user portion 103, locking mechanism 128, the pivot point 109, and the arms 112 are substantially within the noted single plane 146. In a corresponding manner, the normal plane of the projections 113, effectively position the footpads 102 in an offset manner from the noted single plane 146. Rather than lying in a single plane 146, however, some of the noted portions (i.e., the user portion 103, locking mechanism 128, etc.) can be positioned in different, diverging planes (discussed below with regard to FIG. 7).

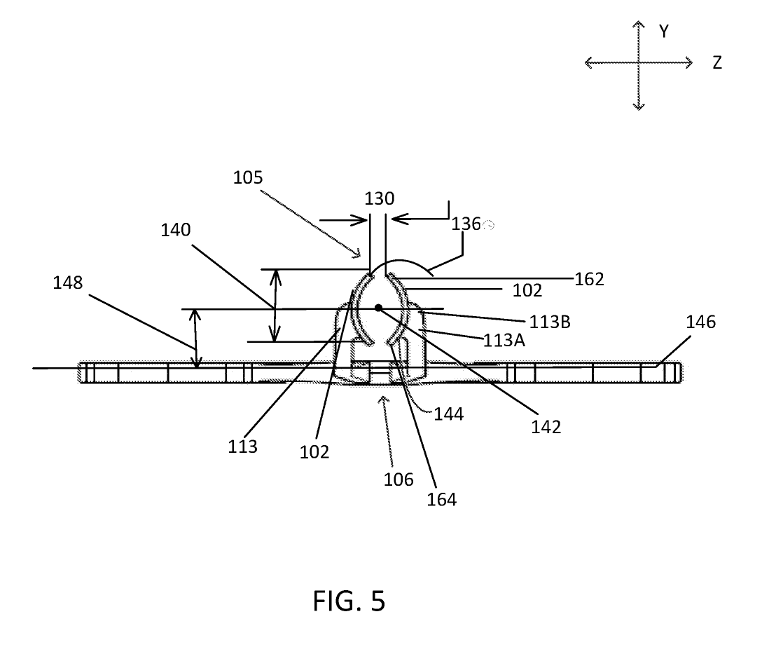

FIG. 5 schematically shows a front view of the tenaculum 100 of FIG. 1. This view better shows how the projections 113 extend out from the noted single plane 146 of the tenaculum 100 to provide the desirable offset for the footpads 102. , More specifically, as shown, the grasping region 105 has an offset (shown at 148) that extends from approximately the central axis 142 running through grasping region 105 (142) to the plane identified in FIG. 5 by 146. As an example, the offset 148 may range from between about 0.5 centimeters and about 2 centimeters. Those skilled in the art can deviate outside of these ranges. In illustrative embodiments, the offset 148 is about 1 centimeter. As noted above, having the grasping region 105 offset from the plane 146 allows other tools (e.g., dilator in FIG. 2) to enter the cervix 116 relatively unobstructed. In some embodiments, the offset 148 is adjustable (e.g., height of projections 113 is adjustable). The offset 148 may cause the bottom ends of the footpads 102 to be similarly offset form the plane 146. As such, the footpads 102 do not intersect the plane 146. Alternative embodiments of the offset 148 may cause the bottom ends of the footpads 102 to end about at or even below the plane 146.

As shown and noted above, the footpads 102 may have an arcuate shape and be mounted on the curved attachment portion 113B of the projections 113. As arcuate members, the footpads 102 can have a radius of curvature 136 extending from a first end 162 to a second end 164. For example, the footpads 102 may have a radius of curvature 136 of about 10 millimeters. Alternatively, the footpads 102 may have different radii of curvature 136 or dimensions. For example, the radius of curvature 136 could be 15 millimeters, 20 millimeters or 30 millimeters. Moreover, when the footpads 102 are in the closed mode, they do not necessarily form a closed circle or oval. Furthermore, various embodiments of the footpads 102 may have different heights 140. For example, the footpads 102 may have heights 140 of approximately 15 millimeters. The height 140 of the footpads 102 should not be confused with the length of the arc of the footpads 102. In illustrative embodiments, the arced footpads 102 have a radius of curvature 136 and extend from between about 15 degrees and about 270 degrees. More preferred embodiments extend from between about 90 degrees and about 180 degrees on the radius of curvature 136.

Of course, the footpads 102 may take on any of a number of different shapes. For example, they may have a varying radius of curvature 136, or be generally flat. Those skilled in the art can select appropriate shape(s), dimension(s) and configuration(s) based on the application.

Illustrative embodiments may be considered to have support surfaces 144 between projections 113 and footpads 102. In some embodiments, the support surfaces 144 may be coupled to and rotate with the adjustable footpads 102 when using such embodiments as discussed above. The support surfaces 144 increase the contact area between the footpads 102 and the projections 113. When the footpads 102 grasp cervical tissue 116, force is felt on the footpads 102. That force is felt at the connection of the footpads 102 and the projections 113. To strengthen the connection between the footpads 102 and the projections 113, the support surfaces 144 provide increase connection area. The increased connection area distributes the force over a larger area, thereby reducing pressure on any given point. Thus, inadvertent failure (i.e., breakage) of the tenaculum 100 (e.g., tenaculums 100 formed from plastic) may be averted. However, support surfaces 144 are not necessary and thus, not intended to limit various embodiments of the invention. For example, in some embodiments, the footpads 102 may be attached to the projections 113 without a support surface 144.

In some embodiments, the footpads 102 may be a removable and/or a disposable component of the tenaculum 100. For example, the footpads 102 could come as fittings sized to be positioned over the pointed ends of conventional single tooth tenaculums. To that end, the fittings may have an open end in which s the pointed ends are positioned. The fittings may be formed from plastic, metal, or any other material suitable to contact with cervical tissue 116 in the medical context. Other than being disposable, the footpads 102 of the fitting may be the same as the above discussed footpads 102. The fittings may come in individually sealed packs, or as a group in containers. A person of skill in the art knows how to size a fitting to fit over a single tooth tenaculum, other conventional tenaculums, and/or forceps. A person of skill in the art also knows how to maintain the orientation of the fitting in relation to the rest of the tenaculum 100 (e.g., pressure fit, snap-on, etc.). Conventional attachment mechanisms, such as a snap fit, may couple the fittings to the conventional tenaculums/devices.

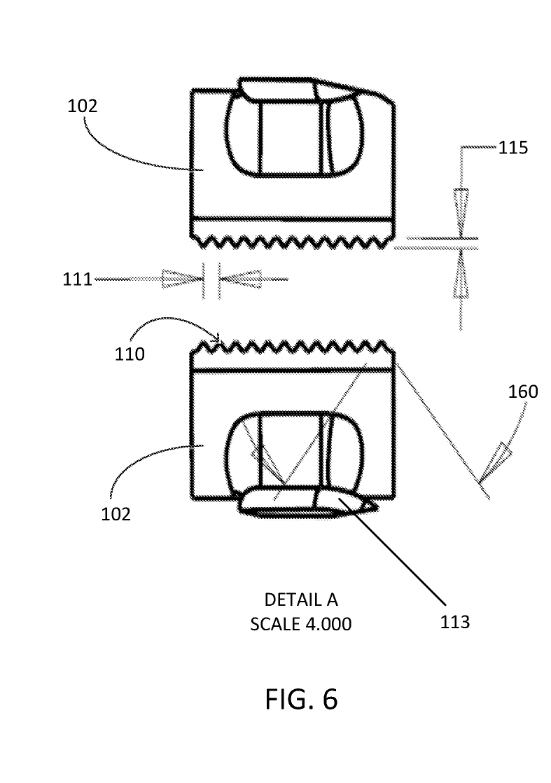

FIG. 6 schematically shows details of the footpads 102 of the tenaculum 100 in FIG. 4. As described above, the shape of the footpads 102 may vary. In illustrative embodiments, the opposing surfaces (also referred to as internal surfaces) of footpads 102 have grasping features 110 to provide better traction and/or grasping of the cervix. In the embodiment shown, ridges form the grasping features 110. In illustrative embodiments, the number and dimensions of ridges 110 may vary. For example, the ridges 110 may extend 115 about 0.25 millimeters from the internal surface of the footpad 102 and may be about 0.5 millimeters wide 111. Additionally, the ridges 110 may form approximately 78.5 degree angles 160. The ridges 110 may take a number of different dimensions and geometries, including but not limited to, recesses, linear structures, and/or bumps. As another example, the grasping features 110 may include a generally roughened and/or frictional surface, with or without ridges 110. In other embodiments, the inner surface of the footpads 102 may be smooth and/or flat.

FIG. 7 schematically shows a perspective view of an alternative embodiment in which the arms 112 of the tenaculum 100 lie in different planes. Unlike the above described embodiments, such as that shown in FIG. 3, the arms 112 have a bend 150 that forms an angle 152 with the longitudinal axis of the tenaculum 100. The bend 150 may enable the practitioner to more easily manipulate the tenaculum 100. In illustrative embodiments, the bend 150 may form a 10 to 20 degree angle 152 (e.g., a 15 degree angle). When the tenaculum 100 is not being manipulated by the practitioner (e.g., it is locked onto the tissue by the locking mechanism 128, it may hang on the grasped tissue. The angle 152 is believed to provide better maneuverability around the device by increasing access to the site of the procedure (compared to some embodiments without the angle 152).

As shown, the proximal end 104, user portion 103 and the portion of the arms 112 up to the bend 150 substantially form a proximal plane 154. In a corresponding manner, the portion of the arms 112 substantially near the distal end 106 form a distal plane 156. This embodiment may share many of the features described above with regard to FIGS. 1-6. For example, in a manner to other embodiments, the projections 113 offset 148 the grasping region 105 from the distal plane 156. Although the tenaculum 100 is shown with just the two planes 154 and 156, illustrative embodiments may form three or more planes. For example, a third plane could be formed by another bend between the proximal plane 154 and the distal plane 156. Alternatively, or additionally, another plane could be formed distal to the distal plane 156. However, it should be understood that, in some embodiments, the distal plane 156 may be the same, or substantially the same, as the plane 146 of the entire tenaculum 100.

Various embodiments of the tenaculum 100 may come in a variety of dimensions and sizes while achieving some advantages of the invention. The various sizes allow physicians to select the most appropriate device for the patient. To that end, various sized tenaculums 100 may be packaged together. For example, six tenaculums 100 may be packaged together in a set. The set may include two different handle styles and each handle style may have three different radii of curvature 136. Some embodiments may include different sized footpads 102. Other embodiments may vary the available contact surface area of the grasping region 105 and/or footpads 102 (e.g., by adjusting the size, shape, location and position of ridges 110). For example, some embodiments of the footpads 102 may be curved to contour the cervix 116, or, as noted above, not be semi-circular in shape. For example, footpads 102 may be rectangular, elliptical, irregularly shaped, or other appropriate shapes. Furthermore, in some embodiments the opposing surface of the footpads 102 may be substantially perpendicular to the distal plane 156, rather than substantially parallel. In some of those embodiments, the central axis 142 of the grasping region 105 may still have an offset 148. In some embodiments, the arms 112 may be flat or angled, or even have just a single arm 112. Illustrative embodiments with a single arm 112 may still have more than one footpad 102 at the grasping region 105.

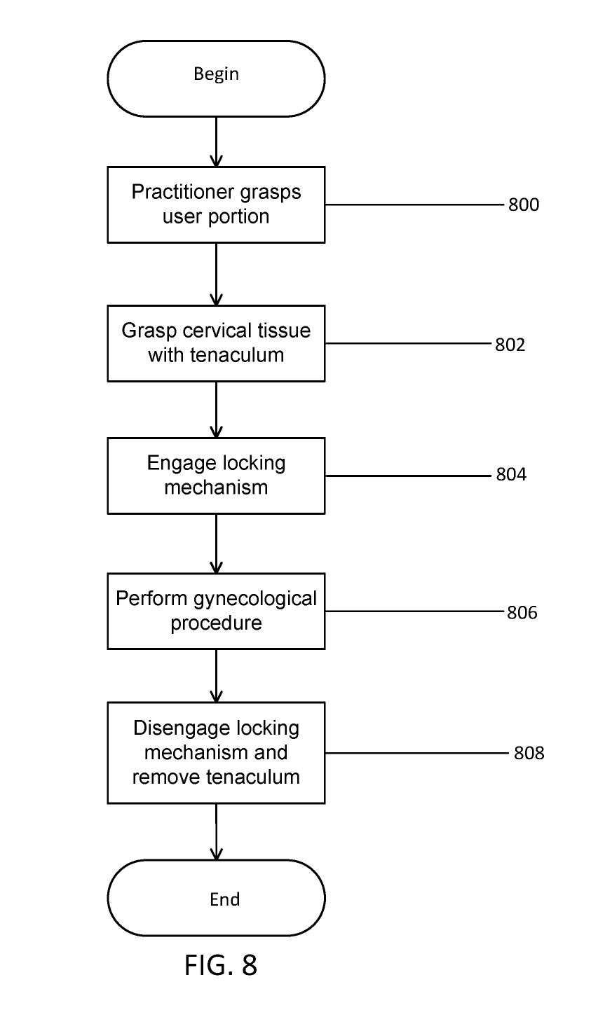

FIG. 8 shows a method of using the tenaculum 100 in accordance with illustrative embodiments of the invention. It should be noted that this process is substantially simplified from a longer process used by a practitioner in performing a relevant medical procedure. Accordingly, the process of using the tenaculum 100 likely has many additional steps, such as patient preparation steps, different in-use activities, and sanitizing steps, which the practitioner likely would perform. In addition, some of the steps may be performed in a different order than that shown, or at the same time. Those skilled in the art therefore can modify the process as appropriate.

The process begins at step 800, in which the practitioner grasps the user portion 103 of the tenaculum 100 and inserts the grasping region 105 into the vagina. Next, at step 802, the practitioner manipulates the user portion 103 to grasp the cervical tissue 116. Specifically, by controlling the user portion 103, the practitioner may rotate the arms 112 relative to each other, about the pivot point 109, to move the first footpad 102 closer to the second footpad 102. This action enables the grasping region 105 to grasp the cervical tissue 116.

The practitioner then may optionally engage the locking mechanism 128 to secure the grasped tissue (step 804). Some embodiments, however, may omit this step and maintain a constant pressure via the user portion 103. The practitioner then may move instruments/tools (e.g., dilator 124) distally into and out of the vagina as part of a medical procedure (e.g., step 806, discussed above with regard to FIG. 2). The process concludes at step 808, in which the practitioner disengages the locking mechanism 128 (if it were engaged), and removes the tenaculum 100 from the vagina.

Although the above discussion discloses various exemplary embodiments of the invention, it should be apparent that those skilled in the art can make various modifications that will achieve some of the advantages of the invention without departing from the true scope of the invention.

What is claimed is:

1. A cervical tenaculum comprising:
a first arm having a proximal end and a distal end;
a second arm having a proximal end and a distal end,
the first arm and the second arm being rotatably connected between the respective proximal and distal ends of the first and second arms, the distal ends of the arms forming an arm plane extending from the distal end;
a user portion at the proximal ends of the first and second arms;
a first projection extending from substantially the distal end of the first arm, the first projection having a first substantially linear portion that diverges from the arm plane;
a second projection extending from substantially the distal end of the second arm, the second projection having a second substantially linear portion that diverges from the arm plane;
a first footpad coupled with the first projection; and
a second footpad coupled with the second projection,
the first and second footpads defining a grasping region for grasping cervical tissue.

2. The cervical tenaculum as defined by claim 1, wherein the first footpad and the second footpad are arcuate shaped.

3. The cervical tenaculum as defined by claim 1, wherein the positions of the first footpad on the first projection and the second footpad on the second projection are fixed.

4. The cervical tenaculum as defined by claim 1, wherein the first projection is rotatably coupled with the first arm, and the second projection is rotatably coupled with the second arm.

5. The cervical tenaculum as defined by claim 1, wherein the first footpad is rotatably coupled with the first projection, and the second footpad is rotatably coupled with the second projection.

6. The cervical tenaculum as defined by claim 1 further comprising a proximal plane formed by the proximal ends of the arms, the proximal plane diverging from the arm plane.

7. The cervical tenaculum as defined by claim 6, wherein the user portion at least in part forms the proximal plane.

8. The cervical tenaculum as defined by claim 1, wherein the footpads are offset from the arm plane.

9. The cervical tenaculum as defined by claim 8, wherein the offset is between about 0.5 centimeter and about 2 centimeters.

10. The cervical tenaculum as defined by claim 1 further comprising a locking mechanism configured to prevent widening or narrowing of the grasping region when engaged.

11. The cervical tenaculum as defined by claim 1, wherein the footpads have tissue contact surfaces,
the tissue contact surfaces further comprising at least one grasping feature.

12. The cervical tenaculum as defined by claim 1, wherein the footpads do not intersect the arm plane.

13. The cervical tenaculum as defined by claim 1, wherein a gap exists between the footpads in a fully closed mode.

14. The cervical tenaculum as defined by claim 1, where the first footpad has a first end and a second end, the first projection being coupled to the first footpad between the first end and the second end.

15. The cervical tenaculum as defined by claim 1, wherein the arm forms an angle of between about 10 and 20 degrees.

16. The cervical tenaculum as defined by claim 1, wherein angles formed between the arms and the substantially linear portions of the projections are adjustable.

17. The cervical tenaculum as defined by claim 1, wherein the length of the projection is adjustable.

18. The cervical tenaculum as defined by claim 1, wherein at least one of the first footpad and the first projection are removable.

19. A cervical tenaculum comprising:
a first arm having a proximal end and a distal end;
a second arm having a proximal end and a distal end,
the first arm and the second arm being rotatably connected between the respective proximal and distal ends of the first and second arms,;
a user portion at the proximal ends of the first and second arms;
a first projection extending from substantially the distal end of the first arm;
a second projection extending from substantially the distal end of the second arm;
a first footpad coupled with the first projection; and
a second footpad coupled with the second projection,
the first and second footpads having opposing surfaces with grasping features for grasping cervical tissue.

20. The cervical tenaculum as defined by claim 19 further comprising a locking mechanism, the locking mechanism configured to prevent rotation of the first arm relative to the second arm when engaged.

21. The cervical tenaculum as defined by claim 19, wherein the distal ends of the arms form an arm plane, further wherein the first projection has a first substantially linear portion that diverges from the arm plane, and the second projection has a second substantially linear portion that diverges from the arm plane.

22. The cervical tenaculum as defined by claim 19, wherein the first footpad and the second footpad are arcuate shaped.

23. The cervical tenaculum as defined by claim 19, wherein the arm forms an angle of between about 10 and 20 degrees.

24. The cervical tenaculum as defined by claim 19, wherein the grasping features comprise ridges.

25. The cervical tenaculum as defined by claim 19, wherein the grasping features comprise a frictional surface.

26. A method of examining a cervix, the method comprising:
providing a cervical tenaculum comprising:
a first arm having a proximal end and a distal end;
a second arm having a proximal end and a distal end,
the first arm and the second arm being rotatably connected between the proximal and distal ends of the first and second arms;
a user portion at the proximal ends of the first and second arms;
a first projection extending from substantially the distal end of the first arm;
a second projection extending from substantially the distal end of the second arm;
a first footpad coupled with the first projection; and
a second footpad coupled with the second projection; and
grasping cervical tissue by rotating the first arm relative to the second arm to move the first footpad closer to the second footpad to grasp the cervical tissue.

27. The method as defined by claim 26 further comprising engaging a locking mechanism to prevent changing the distance between the arcuate footpads by preventing rotation of the first arm relative to the second arm.

28. The method as defined by claim 26 further comprising using the cervical tenaculum to apply a force to the cervical tissue while positioning a second tool into the cervix.

29. The method as defined by claim 26, wherein the cervical tenaculum comprises grasping features on the footpads.

30. The method as defined by claim 26, wherein the first and second arms form an arm plane extending from the distal ends of the first and second arms, the first projection having a first substantially linear portion that diverges from the arm plane.
